(19) United States
(12) Patent Application Publication
(10) Pub. No.: US 2004/0091544 A1
(43) Pub. Date: May 13, 2004

(54) Coated dibasic calcium phosphate

(76) Inventors: Michael D. Ruff, Greenville, NC (US); Joseph E. Cobb JR., Greenville, NC (US)

Correspondence Address: JENNIFER L. SKORD, MOORE & VAN ALLEN, SUITE 800, 2200 WEST MAIN STREET, DURHAM, NC, 27705, US

(21) Appl. No.: 10/643,319

(22) Filed: Aug. 19, 2003

Related U.S. Application Data
(60) Provisional application No. 60/425,024, filed on Nov. 8, 2002.

(51) Int. Cl. A61K 9/16; A61K 38/00; B01J 13/00; A61K 9/50

(52) U.S. Cl. 424/490; 514/2; 427/2.14

(57) ABSTRACT

An oral formulation containing an active pharmaceutical ingredient, for instance a peptide pharmaceutical, such as insulin, coated onto a suitable particulate substrate, such as dibasic calcium phosphate. The oral formulation may be a modified release formulation, for instance a controlled release formulation or a sustained release formulation, or may be an immediate release formulation. Also, the formulation may be encapsulated in gelatin capsules or may be compressed into tablets.

### RELATED APPLICATION

This application is related to U.S. provisional application No. 60/425,024, filed Nov. 8, 2002, entitled “Coated Dibasic Calcium Phosphate”, naming Michael David Ruff and Joseph Earl Cobb, Jr. as the inventors. The contents of the provisional application are incorporated here by reference in their entirety, and the benefit of the filing date of the provisional application is hereby claimed for all purposes that are legally served by such claim for the benefit of the filing date.

### TECHNICAL FIELD

The present invention relates, in general, to pharmaceutical formulations containing an active pharmaceutical ingredient and a non-polysaccharide substrate. More particularly, the present invention relates to an oral formulation containing an active pharmaceutical ingredient, such as a peptide, for instance, insulin, coated onto a non-polysaccharide substrate, such as dibasic calcium phosphate. The oral formulation may be an immediate release formulation or a modified release formulation, such as a sustained release formulation or a controlled release formulation, which two terms are often used interchangeably.

### ABBREVIATIONS

| | |
|---|---|
| active pharmaceutical ingredient | API |
| adenosine triphosphate | ATP |
| buffering agent | BA |
| Centigrade | C |
| cubic centimeter | cc |
| glucose-6-phosphate | G6P |
| glucose-6-phosphate-dehydrogenase | G6PDH |
| gram | g |
| Hertz | Hz |
| hexokinase | HK |
| hexyl insulin monoconjugate-2 | HIM2 |
| hydrophile-lipophile balance | HLB |
| kilogram | kg |
| micrometer | μm |
| milligram | mg |
| milliliter | ml |
| millimeter | mm |
| minute | min |
| nanometer | nm |
| National Formulary | NF |
| nicotinamide adenine dinucleotide | NAD |
| permeation enhancer | PE |
| peroral | po |
| polyethylene glycol | PEG |
| standard deviation | SD |
| weight/weight | w/w |
| Wurster spray granulator | WSG |
| or | |
| Wirbelschict granulator | |
| United States Pharmacopoeia | USP |

### BACKGROUND

Wurster processing in fluid bed units is well recognized for the manufacture of particles containing drug substances (APIs), in an oral dosage formulation. In general, fluid bed processing involves the use of substrates, for instance commercially available polysaccharides such as sugar spheres or spheronized extrudates containing the sugar and the API. Using fluid bed processing, the API may be coated onto the substrate, and a subsequent coating, usually for modification of in vitro release, in vivo release, and/or in vivo absorption, for instance a controlled release material (for instance, an enteric coating material) or a sustained release material, may be applied. Alternatively, the API may be admixed with the substrate, and the resultant made into a spheronized extrudate which is then coated, for instance with a controlled release material or a sustained release material, using fluid bed processing. Coatings are applied as solutions or suspensions, depending on the solubility of the solid components. Also, if an API happens to be a primary amine, a substrate alternative to the sugar typically will be used, since as is well known, an API having a primary amine is typically incompatible with a reducing sugar (e.g, sucrose) due to the Maillard reaction, which causes discoloration.

Oral dosage formulations having an API are desirable in that they afford ease of administration since the patient can swallow the formulation. However, as noted above, in such oral formulations, the API is typically coated onto a polysaccharide substrate, such as commercially available sugar spheres. Alternative substrates are desired for coating with an API due to patient preference or need to avoid sugar-containing products. Also, an alternative may be desired, as sometimes there may be poor adherence of the coating material to the substrate.

More specifically, diabetics, particularly diabetics who have Type 1 diabetes and sometimes diabetics who have Type 2 diabetes, inject the peptide, insulin, for maintaining their blood sugar at a normal level. As is well known, injecting the insulin is very undesirable for a various reasons, such as the pain caused by needles, the soreness caused by needles, aversion by the patient to injecting himself/herself with a needle, and so on. The current commercial oral dosage formulations for diabetics do not contain insulin.

Thus, oral dosage formulations having insulin as the API would be desirable so that the patient could swallow the insulin instead of injecting it. However, as noted above, in oral formulations where an API is coated onto a substrate, the substrate is typically a polysaccharide substrate, such as a commercially available sugar spheres. Thus, a substrate alternative to a polysaccharide is particularly desired for coating with insulin due to patient preference or need to avoid sugar-containing products.

Hence, a need exists for APIs, for instance a peptide such as insulin, to be coated onto a substrate that is free of polysaccharides in order to provide a formulation for oral administration to patients, especially those patients who are diabetics.

### SUMMARY OF THE INVENTION

Accordingly, the present invention provides a process for making a pharmaceutical formulation for oral administration comprising forming a solution of an active pharmaceutical ingredient and applying the resultant solution to form a coating on a particulate pharmaceutical substrate, wherein the substrate is free of a polysaccharide. Preferably, the substrate comprises a particulate calcium pharmaceutical substrate, wherein the substrate is free of a polysaccharide. Also preferably, the active pharmaceutical ingredient comprises a peptide.

In a preferred embodiment, the present invention provides a process for making a pharmaceutical formulation for oral administration of insulin comprising applying a solution of an insulin to form a coating on a particulate calcium pharmaceutical substrate, wherein the substrate is free of a polysaccharide. More preferably, the particulate calcium substrate has been coated with a permeation enhancer.

Also, the present invention provides an oral pharmaceutical formulation comprising a particulate pharmaceutical substrate having an application of an active pharmaceutical ingredient coating, wherein the substrate is free of a polysaccharide. Preferably, the substrate comprises a particulate calcium pharmaceutical substrate, wherein the substrate is free of a polysaccharide. Also preferably, the active pharmaceutical ingredient comprises a peptide. Typically, the API is present in a load from about 0.1% to about 30% w/w.

In a preferred embodiment, the present invention provides a pharmaceutical formulation for oral administration of insulin comprising a particulate calcium pharmaceutical substrate having an application of an insulin coating, wherein the particulate calcium substrate is free of a polysaccharide. More preferably, the particulate calcium pharmaceutical substrate also has an application of a permeation enhancer coating, and the insulin coating has been applied over the permeation enhancer coating. Typically, the insulin is present in a load from about 0.1% to about 30% w/w.

Thus, it is an object of the invention to avoid polysaccharide substrates, such as sugar, for use in pharmaceutical formulations that are to be orally administered to the patient.

Other features, aspects, and advantages of the present invention will become better understood with reference to the following description, accompanying Figures, accompanying Laboratory Examples, and appended claims.

### BRIEF DESCRIPTION OF THE DRAWINGS

FIG. 1 is a photograph taken through a microscope of particulate sugar spheres (non-pareils), 30/35 mesh sieve cut, at 150× magnification.

FIG. 2 is a photograph taken through a microscope of CELPHERE® CP-507 (microcrystalline cellulose) at 150× magnification.

FIG. 3A is a photograph taken through a microscope of AVICEL® PH 200 (microcrystalline cellulose) at 150× magnification.

FIG. 3B is a photograph taken through a microscope of AVICEL® PH 200 (microcrystalline cellulose) at 200× magnification.

FIG. 4A is a photograph taken through a microscope of EMCOMPRESS® (dicalcium phosphate dihydrate) at 150× magnification.

FIG. 4B is two photographs taken through a microscope of EMCOMPRESS® (dicalcium phosphate dihydrate) at 50× magnification and 200× magnification.

FIG. 5 is a bar graph depicting the sonic sieve analysis of AVICEL® PH 200 and of AVICEL® PH 200 coated with permeation enhancers.

FIG. 6 is a bar graph depicting the sonic sieve analysis of EMCOMPRESS® and of EMCOMPRESS® coated with permeation enhancers.

FIG. 7 is a photograph taken through a microscope of EMCOMPRESS® formulation 1 at 150× magnification.

FIG. 8 is a photograph taken through a microscope of EMCOMPRESS® formulation 2 at 150× magnification.

FIG. 9 is a photograph taken through a microscope of EMCOMPRESS® formulation 3 at 150× magnification.

FIG. 10 is a graph showing the change in glucose level for each of 4 dogs tested with formula 2 (second sample) from Table C.

FIG. 11 is a graph showing the change in glucose level for each of 4 dogs tested with formula 3 from Table C.

FIG. 12 is a graph showing the change in glucose level for each of 4 dogs tested with sample 1, white tablet with no enteric coating from Table F.

FIG. 13 is a graph showing the change in glucose level for each of 4 dogs tested with sample 1, white tablet with enteric coating at pH 5.5 from Table F.

FIG. 14 is a graph showing the change in glucose level for each of 4 dogs tested with sample 2, blue tablet with no enteric coating from Table F.

FIG. 15 is a graph showing the change in glucose level for each of 4 dogs tested with sample 2, blue tablet with enteric coating at pH 7.0 from Table F.

FIG. 16 is a graph showing the change in glucose level for each of 4 dogs tested with sample 3, red tablet with no enteric coating from Table F.

FIG. 17 is a graph showing the change in glucose level for each of 4 dogs tested with sample 3, red tablet with enteric coating at pH 7.0 from Table F.

FIG. 18 is a graph showing the change in glucose level for each of 4 dogs tested with sample 4, yellow tablet with no enteric coating from Table F.

FIG. 19 is a graph showing the change in glucose level for each of 4 dogs tested with sample 4, yellow tablet with enteric coating at pH 5.5 from Table F.

FIG. 20 is a graph showing the change in glucose level for each of 4 dogs tested with sample 5, orange tablet with no enteric coating from Table F.

FIG. 21 is a graph showing the change in glucose level for each of 4 dogs tested with sample 5, orange tablet with enteric coating at pH 7.0 from Table F.

FIG. 22 is a graph showing the change in glucose level for each of 4 dogs tested with sample 6, green tablet with no enteric coating from Table F.

FIG. 23 is a graph showing the change in glucose level for each of 4 dogs tested with sample 6, green tablet with enteric coating at pH 5.5 from Table F.

FIG. 24 is a graph showing the change in glucose level for each of 4 dogs tested with sample 7, purple tablet with no enteric coating from Table F.

FIG. 25 is a graph showing the change in glucose level for each of 4 dogs tested with sample 7, purple tablet with enteric coating at pH 5.5 from Table F.

There is no graph for sample 8, dark red tablet with no enteric coating from Table F, as this tablet was not tested in dogs.

FIG. 26 is a graph showing the change in glucose level for each of 4 dogs tested with sample 8, dark red tablet with enteric coating at pH 7.0 from Table F.

### DETAILED DESCRIPTION

The term “solution” as used here is meant to refer to true solutions as well as to suspensions where a dispersing agent, an emulsifier, a surfactant, or the like is included to maintain the components in an admixed state.

A suitable substrate should be chosen so that it is free of polysaccharide. Also desirably, the substrate should be a particulate that physically resembles sugar sphere particulates that are commonly used as substrates. Thus, the substrate should be a particulate of generally spheroid shape, have a bulk density similar to that of sugar spheres, have a particle size similar to that of sugar spheres, and have a relatively narrow range of particle size in order to obviate performing a sieve cut. Also, the substrate particles should have a porous surface, which is preferred over a smooth surface.

Suitable particulate substrates include, but are not limited to, calcium materials. Suitable calcium materials include calcium phosphate, dibasic (also called dicalcium phosphate or dicalcium phosphate dihydrate or dibasic calcium phosphate dihydrate). Although calcium phosphate, dibasic is preferred, also useful are monobasic calcium phosphate, monohydrate (also called monocalcium phosphate or monobasic calcium phosphate), or tribasic calcium phosphate (also called tricalcium phosphate). The anhydrous form of dibasic calcium phosphate is also useful. Also, calcium carbonate or calcium citrate should work. Additionally, calcium sulfate (either anhydrous or dihydrate), which is typically available as a fine powder, could be modified by wet granulation to form a generally spherical substrate which could then be coated. The least preferred of the calcium materials for use as a substrate probably is calcium stearate because, although it is commercially available as a pharmaceutically acceptable calcium salt (USP/NF Compendia), it is a waxy lubricant material that may not agglomerate and coat as well as desired.

Suitable dicalcium phosphate dihydrate is commercially available as EMCOMPRESS® from Penwest, Incorporated, CALSTAR® from Astaris LLC Limited Liability Corporation, and DI-TAB® from Rhodia Incorporated, assignee of Stauffer Chemical Company Corporation. Suitable dibasic calcium phosphate anhydrous is commercially available as EMCOMPRESS® Anhydrous from Edward Mendell Company Incorporated and A-TAB® from Rhone-Poulenc Chemicals Company. Suitable tricalcium phosphate is commercially available as TRI-TAB® and TRI-CAL®, each from Rhone-Poulenc Chemicals Company, assignee of Stauffer Chemical Company Corporation. It is noted that Edward Mendell Company Incorporated was absorbed by Penwest, Incorporated, which was then absorbed by JRS Pharma LP (J. Rettenmaier and Sohne GmbH) who is now the owner of the EMCOMPRESS® products. Calcium sulfate powder is marketed as CAL-TAB® from Ingredient Technology Corporation, COMPACTROL® from Edward Mendell Company Incorporated, and DESTAB® from K-V Pharmaceutical Company Corporation.

All of these calcium materials should have a porous surface. Microcrystalline cellulose material, such as that sold by FMC Corporation under the trademark AVICEL®, has a porous surface and thus should also be useful as a substrate.

The substrate may be coated with one or more coatings. At least one of the coatings must comprise an API, preferably a peptide pharmaceutical as an API.

A suitable peptide pharmaceutical is insulin. The insulin may be the human kind, which is synthesized from a non-disease producing laboratory strain of Escherichia coli bacteria that has been genetically altered by the addition of the gene for human insulin production, or the insulin may be the animal kind, which comes from pigs.

Particularly, hexyl insulin monoconjugate-2 polydisperse (abbreviated here as HIM2 polydisperse) is a very suitable for the peptide pharmaceutical for use as an API. HIM2 polydisperse is a human insulin that is PEGylated (i.e., reacted to have polyethylene glycol moieties) with PEG 400. PEG 400 is so designated as it is a PEG with a molecular weight of 400. PEG 400 is a viscous liquid at room temperature. The PEG 400 is attached to sites on the insulin where enzymatic attack of the insulin normally occurs, and thus protects the insulin from degradation. Also, the PEG 400 has a HLB-raising effect, and thus is believed to improve the solubility of the insulin. HIM2 polydisperse has a molecular weight of approximately 6200 (5800 from insulin and 400 from PEG).

The coating of the API, for instance a peptide pharmaceutical, may be applied to the particulate substrate as a solution, for instance, an aqueous solution. The solution may be sprayed onto the substrate, and equipment, such as a fluid bed processor with a spray granulator, is well known to those of ordinary skill in the art for spraying various coatings onto substrates for pharmaceutical products. The solution may be applied to the substrate by mixing in a blender, and blender equipment is well known to those of ordinary skill in the art for applying coatings onto substrates for pharmaceutical products.

The coating of the API should be applied to the particulate substrate to achieve an API load ranging from about 0.1% to about 30% w/w, more preferably about 0.5% to about 24% w/w, more preferably about 0.8% to about 13% w/w, more preferable about 0.9% to about 5%, and most preferably about 1% to about 2% w/w.

There may be one or more other coatings applied before and/or after the active pharmaceutical ingredient coating. Hence, one or more coatings may be under the active pharmaceutical ingredient coating and/or one or more coatings may be over the active pharmaceutical ingredient coating.

The other coatings, or other materials besides the API in the active pharmaceutical ingredient coating, may be any of the typical materials employed in pharmaceuticals as coating agents, modified release agents such as controlled release agents (for instance, enteric agents) or sustained release agents, and/or excipient agents. Such materials include, but are not limited to, BAs, PEs, colorants, film-forming polymers, plasticizers, surfactants, dispersions of ethyl cellulose, coating lacquers, pigments, and the like.

Suitable BAs include, but are not limited to, citric acid, heptahydrate sodium phosphate, and combinations thereof.

Suitable PEs include, but are not limited to, sodium cholate, lauric acid, oleic acid, capric acid, and the sodium salts of the acids, namely sodium laurate, sodium oleate, sodium caprate, and combinations thereof. Particularly, when insulin is the API, it is believed that these PEs possess a micelle-forming action that improves solubility and permeability.

To help obviate the problem of fracturing the coating(s) that may occur if the particles are compressed into tablets, the particulate pharmaceutical substrate having an application of an active pharmaceutical ingredient coating, such as a peptide pharmaceutical coating, may be encapsulated in a gelatin capsule. Suitable gelatin capsules are sold under the trademark CAPSUGEL® by Warner-Lambert Company. Shionogi also sells suitable gelatin capsules.

### LABORATORY EXAMPLES

Materials. The following materials were employed in making the below-described Examples A, B, and C.

Sugar spheres (non-pareils) 30/35 mesh cut were supplied by Paulaur Corporation. EMCOMPRESS® was supplied by Penwest, Incorporated. (EMCOMPRESS® is a trademark of Edward Mendell Company for dicalcium phosphate dihydrate, USP.) AVICEL® PH 200 and AVICEL® PH 102 were supplied by FMC Corporation. (AVICEL® is a trademark of FMC Corporation for microcrystalline cellulose, NF.) CELPHERE® CP-507 beads were supplied by Asahi Kasei Company. (CELPHERE® is a trademark of Asahi Kasei Corporation for microcrystalline cellulose for use in the manufacture of pharmaceuticals.)

HIM2 polydisperse (insulin modified with a polyethylene glycol type ester to help prevent the insulin from being cleaved by stomach enzymes), permeation enhancers (sodium cholate, lauric acid, oleic acid, capric acid, and the sodium salts of the acids, namely sodium laurate, sodium oleate, sodium caprate), and buffering agents (citric acid and sodium phosphate heptahydrate and sodium hydroxide) were supplied by Nobex Corporation.

SURELEASE® was supplied by Colorcon, Incorporated. (SURELEASE® is a trademark of Colorcon, Incorporated for polymeric dispersions of ethyl cellulose for use as sustained release film coatings for pharmaceutical products.)

OPADRY® II Clear YS-1-7006 was supplied by Colorcon, Incorporated. (OPADRY® is a trademark of Colorcon, Incorporated for edible film-forming polymers, plasticizers, and surfactants, with or without pigments, for use in pharmaceuticals for coating tablets.)

EUDRAGIT® L30D-55 and EUDRAGIT® RS30D were supplied by Rohm Pharma Polymers, Degussa. (EUDRAGIT® is a trademark of Rohm & Haas GmbH Company for coating lacquers used on medicinal tablets.)

CAPSUGEL® gelatin capsules were supplied by Warner-Lambert Company. (CAPSUGEL® is a trademark of Warner-Lambert Company for empty gelatin capsules).

CAB-O-SIL® was supplied by Cabot Corporation, Boston, Mass., U.S.A. (CAB-O-SIL® is a trademark of Cabot Corporation for fumed silicon dioxide anti-caking agent.)

Equipment. The following equipment was employed in making the below-described Examples A, B, and C.

A UNIGLATT (UNIGLATT is a trade name and GLATT® is a trademark of Glatt GmbH, a limited joint stock company in the Federal Republic of Germany; glatt is the German word for smooth) fluid bed processor WSG-1 (blender/coater/sprayer device) with a variable frequency drive for the turbine was obtained from Glatt GmbH. The processing insert was a standard 6 inch Wurster (trade name) spray granulator.

A MASTERFLEX® (trademark of Cole-Parmer Instrument & Equipment Company for fluid pumps and for flexible tubing for use with fluid pumps) peristaltic pump equipped with MASTERFLEX® size number 14 silicone tubing was obtained from Masterflex.

A GILSONIC® (trademark of Gilson Company for an ultrasonic vibrating sieve shaker) auto siever equipped with various screens was obtained from Gilson Company. Also used was a light microscope with a camera mount.

Evaluation of substrate candidates. Three particulate substrates, each free of polysaccharide, were chosen for evaluation. They were (A) CELPHERE CP-507 (microcrystalline cellulose), (B) AVICEL PH 200 (microcrystalline cellulose), and (C) EMCOMPRESS (dicalcium phosphate dihydrate).

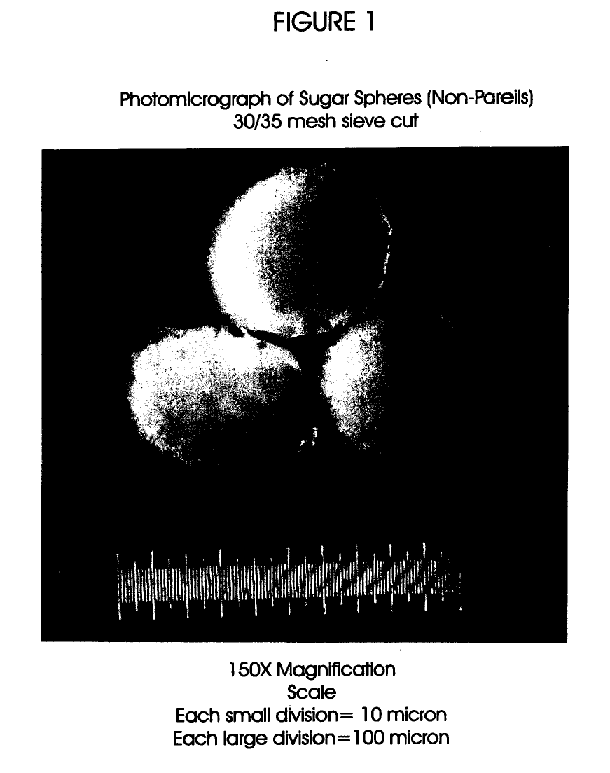

As shown in the photographs in FIGS. 1, 2, 3A, 3B, 4A, and 4B, sugar spheres and all three substrate candidates were examined under a light microscope with mineral oil to compare morphology of the three substrate candidates to that of sugar spheres vis-à-vis spheroid shape, bulk density similar to sugar spheres, particle size similar to sugar spheres, and relatively narrow range of particle size (without having to perform a sieve cut).

Additionally, bulk density, tapped density, flow (Carr's compressibility index), and sonic sieve were determined for each of the three substrate candidates, CELPHERE CP-507 (microcrystalline cellulose), AVICEL PH 200 (microcrystalline cellulose), and EMCOMPRESS (dicalcium phosphate dihydrate).

Bulk density was performed in duplicate by carefully loading (with minimum vibration) glass graduated cylinders approximately half full by volume for each of the three substrates, weighing the sample and reading the volume. Values are a mean of n=2.

Tapped density was performed in duplicate for each of the three substrates using a VanKel Tap Density Tester, with 50 taps per analysis. Values are a mean of n=2.

Carr's compressibility index (from R. L. Carr, Brit. Chem. Eng., (1970) pp. 1541-1549), which is an approximate measure of flow, was calculated using the formula: Carr's Index=(tapped density−bulk density)÷tapped density×100%. In accordance with the system of Carr, the following relationship applies: 5 to 15% indicates excellent flow, 12 to 16% indicates good to fair flow, 23 to 35% indicates poor flow, 33 to 38% indicates very poor flow, and >40% indicates, very, very poor flow.

Sonic sieve was determined in that a 10 gram quantity of each of CELPHERE CP-507 (microcrystalline cellulose), AVICEL PH 200 (microcrystalline cellulose), and EMCOMPRESS (dicalcium phosphate dihydrate) was sonic-sifted in the GILSONIC auto siever equipped with 20 mesh (850 μm), 40 mesh (425 μm), 60 mesh (250 μm), 80 mesh (180 μm), 100 mesh (150 μm), and 200 mesh (75 μm) screens and a fines collector. Samples were sifted for 6 minutes using a 2 minute ramp up interval, followed by a 2 minute constant pulse interval, and a final 2 minute ramp down cycle, with 50% amplitude. The % retained on each screen was recorded.

The bulk density, tapped density, Carr's compressibility index (flow), and sonic sieve results are summarized in the following Tables 1, 2, 3, and 4.

TABLE 1

| Substrate | Sugar spheres |
|---|---|
| | (30/35 sieve mesh cut) |
| Gross morphology (see, photograph taken | Spherical, smooth surface |
| through microscope, FIG. 1) | |
| Bulk density (g/cc) | 0.80 |
| Tapped density (g/cc) | 0.88 |
| Carr's Compressibility Index (%) | 9.1 (excellent) |
| (measure of flow) | |
| Sonic sieve (% retained on 20 mesh screen) | 0 |
| Sonic sieve (% retained on 40 mesh screen) | 99.6 |
| Sonic sieve (% retained on 60 mesh screen) | 0.4 |
| Sonic sieve (% retained on 80 mesh screen) | 0 |
| Sonic sieve (% retained on 100 mesh screen) | 0 |
| Sonic sieve (% retained on 200 mesh screen) | 0 |
| Sonic sieve (% fines) | 0 |

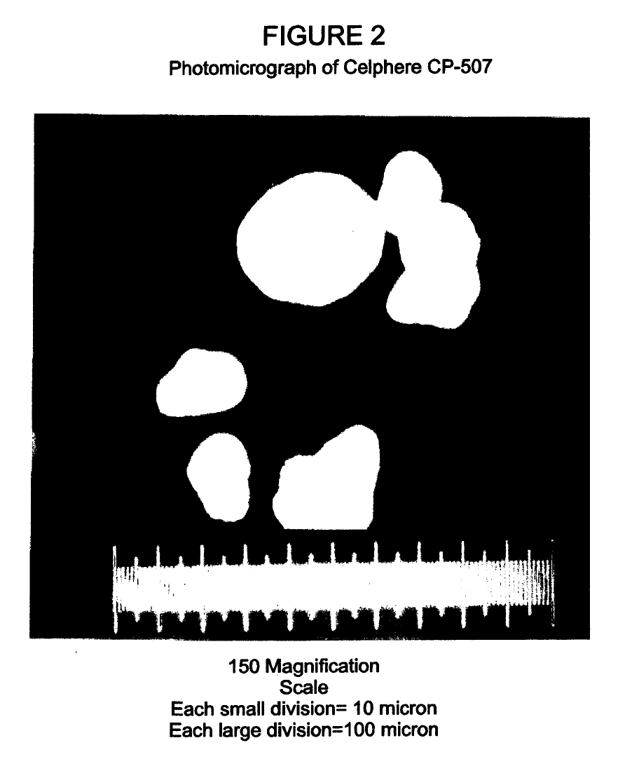

TABLE 2

| Substrate | CELPHERE CP-507 ® |
|---|---|
| | (microcrystalline cellulose) |
| Gross morphology (see, photograph taken | Spheroid, smooth surface |
| through microscope, FIG. 2) | |
| Bulk density (g/cc) | 0.97 |
| Tapped density (g/cc) | 1.09 |
| Carr's Compressibility Index (%) | 11.0 (excellent) |
| (measure of flow) | |
| Sonic sieve (% retained on 20 mesh screen) | 0 |
| Sonic sieve (% retained on 40 mesh screen) | 2.6 |
| Sonic sieve (% retained on 60 mesh screen) | 74.0 |
| Sonic sieve (% retained on 80 mesh screen) | 15.1 |
| Sonic sieve (% retained on 100 mesh screen) | 4.6 |
| Sonic sieve (% retained on 200 mesh screen) | 3.7 |
| Sonic screen (% fines) | 0 |

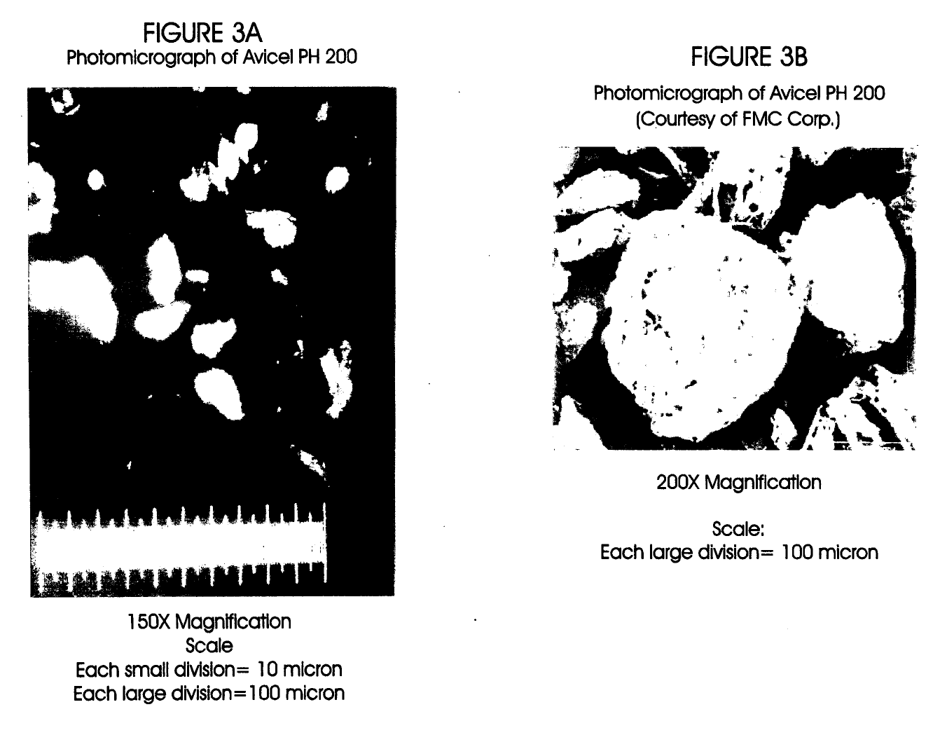

TABLE 3

| | AVICEL ® PH 200 |
|---|---|
| Substrate | (microcrystalline cellulose) |
| Gross morphology (see, photographs taken | Irregular spheroid, coarse, |
| through microscope, FIGS. 3A and 3B) | porous surface |
| Bulk density (g/cc) | 0.36 |
| Tapped density (g/cc) | 0.44 |
| Carr's Compressibility Index (%) | 18.2 (passable/fair) |
| (measure of flow) | |
| Sonic sieve (% retained on 20 mesh screen) | 0 |
| Sonic sieve (% retained on 40 mesh screen) | 0.1 |
| Sonic sieve (% retained on 60 mesh screen) | 15.0 |
| Sonic sieve (% retained on 80 mesh screen) | 28.6 |
| Sonic sieve (% retained on 100 mesh screen) | 9.8 |
| Sonic sieve (% retained on 200 mesh screen) | 27.0 |
| Sonic screen (% fines) | 19.5 |

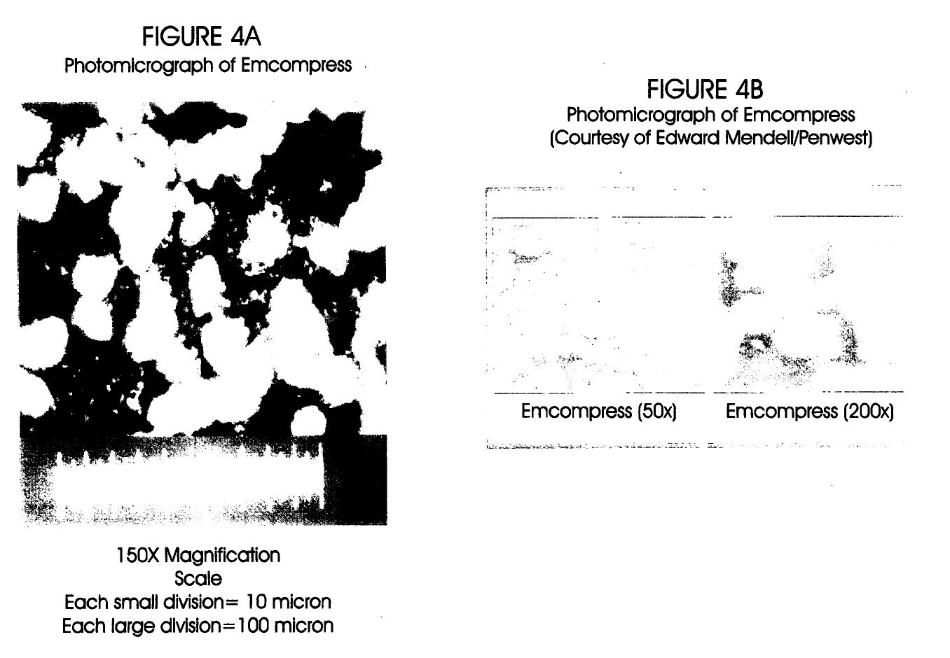

TABLE 4

| Substrate | EMCOMPRESS ® |
|---|---|
| | (dicalcium phosphate |
| | dihydrate) |
| Gross morphology (see, photographs taken | Irregular spheroid, |
| | coarse porous |
| through microscope, FIGS. 4A and 4B) | surface |
| Bulk density (g/cc) | 0.88 |
| Tapped density (g/cc) | 1.07 |
| Carr's Compressibility Index (%) | 17.8 (fair/good) |
| (measure of flow) | |
| Sonic sieve (% retained on 20 mesh screen) | 0 |
| Sonic sieve (% retained on 40 mesh screen) | 0.1 |
| Sonic sieve (% retained on 60 mesh screen) | 17.6 |
| Sonic sieve (% retained on 80 mesh screen) | 40.8 |
| Sonic sieve (% retained on 100 mesh screen) | 17.3 |
| Sonic sieve (% retained on 200 mesh screen) | 20.8 |
| Sonic sieve (% fines) | 3.3 |

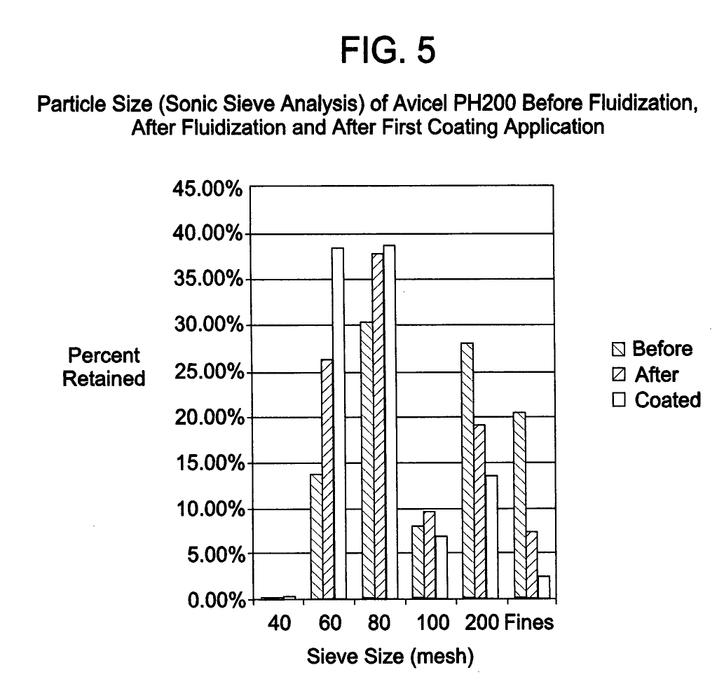

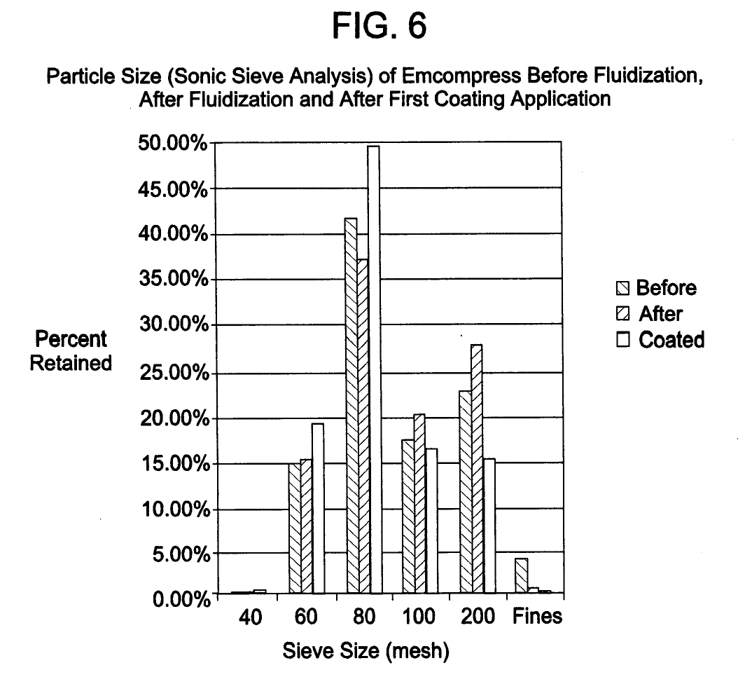

Furthermore, some of the below described coated substrates were sonic-sifted in the GILSONIC auto siever equipped with the various mesh screens for comparison to uncoated materials, as depicted in the bar graphs of FIGS. 5 and 6.

Processing parameters. The processing parameters using the UNIGLATT fluid bed processor WSG-1 with 6 inch Wurster in each of the below described Examples A, B, and C were as follows. The inlet air temperature was from about 70° C. to about 80° C. The spray rate was from about 4 to about 10 g/min. The variable frequency drive was from about 15 to about 40 Hz (about 25% to about 67% of relative turbine capacity). The partition height was from about 12 to about 20 mm. The atomization air pressure was from about 3 to about 4 bar.

### Example A

CELPHERE CP-507 (microcrystalline cellulose) was employed as the particulate substrate. Samples were prepared as follows.

A buffered aqueous solution of PE (approximately 29% solids) was prepared and sprayed onto CELPHERE CP-507 (microcrystalline cellulose) using the UNIGLATT fluid bed processor WSG-1 with 6 inch Wurster.

The PE coating did not adhere well for making CELPHERE/PE. It is believed this adherence problem occurred because, as was seen from examining CELPHERE under the light microscope, the surface of the CELPHERE beads was very smooth (not porous like the surface of each of AVICEL PH 200 and EMCOMPRESS when observed under the light microscope), which probably prevented successful buildup of a coating on CELPHERE. Adjustments to the processing parameters for the spray rate, inlet temperature, and air volume did not appear to correct the adherence problem. Also, trying addition of POVIDONE sticky binder did not appear to correct the adherence problem to CELPHERE.

Hence, CELPHERE was abandoned as a suitable alternative substrate to sugar spheres and no attempts were made to coat the CELPHERE/PE with an API, such as insulin.

### Example B

AVICEL PH 200 (microcrystalline cellulose) was employed as the particulate substrate. Samples were prepared as follows.

The same buffered aqueous solution of PEs (approximately 29% solids) as employed above in Example A was sprayed onto 1000-1500 g of AVICEL using the UNIGLAT fluid bed processor WSG-1 with 6 inch Wurster.

AVICEL coated well with PEs. More specifically, the PE coating on the AVICEL was demonstrated by performing a sonic sieve analysis with the GILSONIC on the AVICEL/PE particles, and a shift was observed as compared to the sonic sieve analysis with the GILSONIC of the AVICEL particles. See the bar graph in FIG. 5.

However, significant fines were generated during coating, resulting in product loss through the 20 μm filter bag (fines collector) of the WSG-1. There was significant loading of filter bags, apparently resulting from the low bulk density of the AVICEL/PE particles. The fines were likely the result of poor friability of the AVICEL/PE particles, which appeared not to be able to withstand the rigors of the relatively slow Wurster column process.

Although no attempts were made to coat the AVICEL/PE with insulin, the AVICEL/PE is believed to be suitable for coating with an API, such as insulin.

### Example C

EMCOMPRESS (dicalcium phosphate dihydrate) was employed as the particulate substrate. Samples were prepared as follows.

The same buffered aqueous solution of PEs (approximately 29% solids) as employed above in Example A was sprayed onto 1000-1500 g of EMCOMPRESS using the UNIGLATT fluid bed processor WSG-1 with 6 inch Wurster.

EMCOMPRESS accepted the PE coat well with minimal product loss. No significant agglomeration was encountered. EMCOMPRESS/PE particles visually appeared uniform from observations through the light microscope. Also, build-up of the PE coating on the EMCOMPRESS was demonstrated by performing a sonic sieve analysis with the GILSONIC on the EMPRESS/PE particles, and a shift was observed as compared to the sonic sieve analysis with the GILSONIC of the EMPRESS particles. See the bar graph in FIG. 6.

Coating of the EMCOMPRESS/PE particles with insulin as an API was performed for the following various formulae.

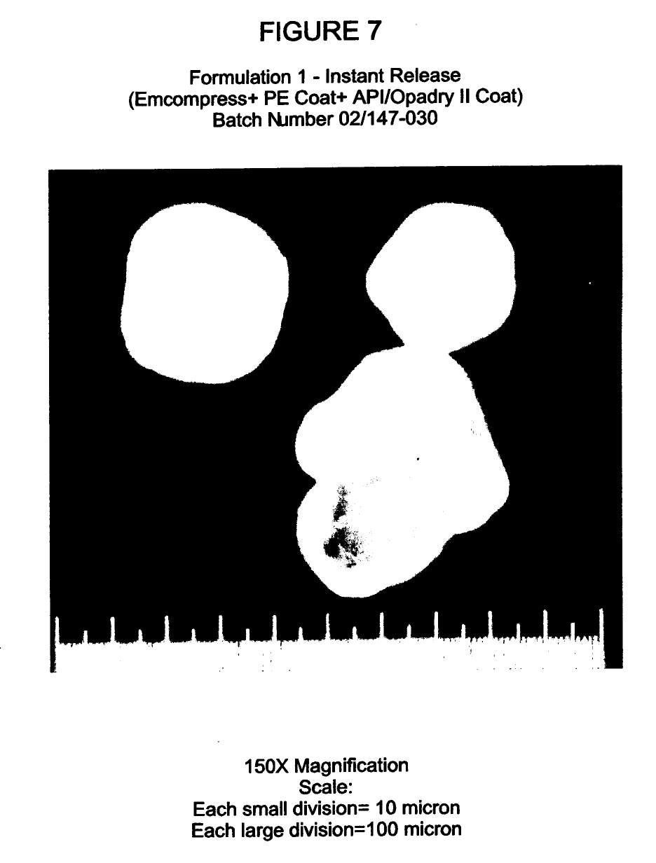

Formula 1 (first sample, second sample, and third sample), EMCOMPRESS and HIM2 polydisperse (instant release, also called immediate release). A selected amount of the EMCOMPRESS/PE particles were coated as follows. An aqueous solution of approximately 10% w/w of HIM2 polydisperse (modified insulin, available from Nobex Corporation) and approximately 8% w/w of OPADRY II Clear YS-1-7006 (edible film-forming polymers, plasticizers, and surfactants) was prepared, and then sprayed onto the EMCOMPRESS/PE particles to a weight gain achieving a drug load of about 1% to about 2% w/w, making three variations. The EMCOMPRESS/PE accepted the insulin coating well. See the photograph in FIG. 7. Also see formula 1 (first sample, second sample, and third sample) in Table C below.

Then, selected EMCOMPRESS/PE/insulin particles were coated with two different modified release agent systems, described below as formula 2 and formula 3.

Formula 2 (first sample and second sample), EMCOMPRESS and HIM2 polydisperse (controlled release). A selected amount of the EMCOMPRESS/PE/insulin particles were coated as follows. An enteric (pH dependent) system using EUDRAGIT L30D-55 (coating lacquer), triethyl citrate, and talc was prepared, and then was sprayed onto the EMCOMPRESS/PE/insulin particles. See formula 2 (first sample) in Table C below.

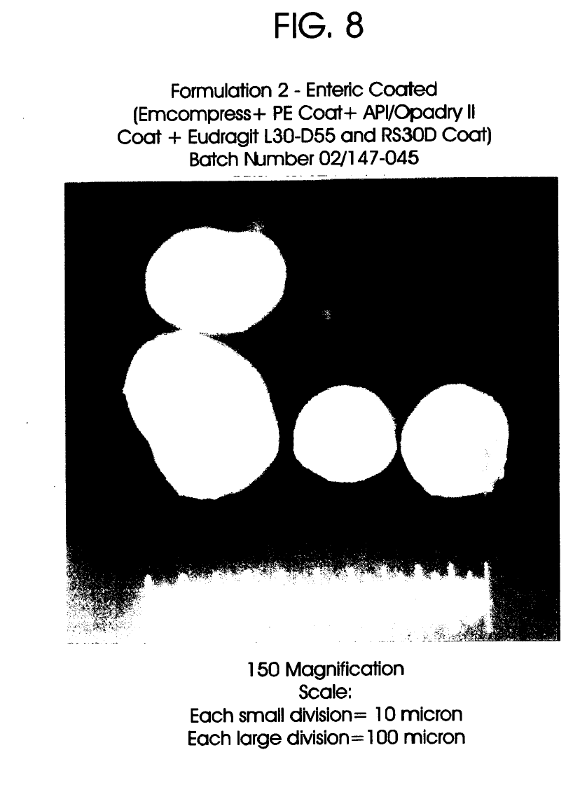

Based on preliminary evaluations, the particles coated with the same ingredients were again coated, this time using EUDRAGIT RS30D (coating lacquer), acetyl tributyl citrate, and talc. Particles were coated to a weight gain of about 6% for each EUDRAGIT. The EMCOMPRESS/PE/insulin accepted the EUDRAGIT coatings well. See the photograph in FIG. 8. Also see formula 2 (second sample) in Table C below.

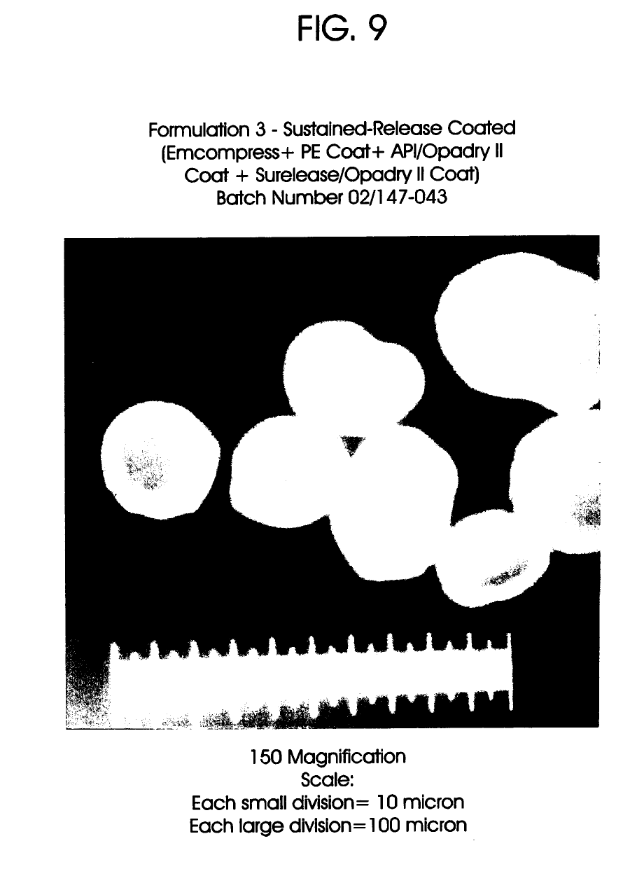

Formula 3 (only sample), EMCOMPRESS and HIM2 polydisperse (sustained release). A selected amount of the EMCOMPRESS/PE/insulin particles were coated as follows. A sustained-release system containing SURELEASE (dispersion of ethyl cellulose) and OPADRY II Clear YS-1-7006 (edible film-forming polymers, plasticizers, and surfactants) was prepared, and then sprayed onto the EMCOMPRESS/PE/insulin particles to a weight gain of about 4 to about 9% of SURELEASE and a weight gain of about 2% of OPADRY. The EMCOMPRESS/PE/insulin accepted the SURELEASE+OPADRY coating well. See the photograph in FIG. 9. Also see formula 3 in Table C below.

TABLE C

| | Formulae of EMCOMPRESS and HIM2 polydisperse* | | | | | |
|---|---|---|---|---|---|---|
| | formula | formula | formula | formula | formula | formula |
| | 1 | 1 | 1 | 2 | 2 | 3 |
| INGRE- | (first | (second | (third | (first | (second | (only |
| DIENTS | sample) | sample) | sample) | sample) | sample) | sample) |
| HIM2 | 1.08 | 20.0 | 6.0 | 12.9 | 11.6 | 5.8 |
| polydisperse | | | | | | |
| insulin | | | | | | |
| EM- | 65.56 | 445.3 | 216.3 | 286.4 | 259.0 | 209.9 |
| COMPRESS | | | | | | |
| (dicalcium | | | | | | |
| phosphate | | | | | | |
| dihydrate) | | | | | | |
| capric acid | 2.52 | 48.4 | 23.6 | 31.1 | 28.1 | 22.9 |
| citric acid | 3.49 | 99.1 | 48.1 | 63.7 | 57.6 | 46.6 |
| lauric acid | 3.49 | 99.1 | 48.1 | 63.7 | 57.6 | 46.6 |
| oleic acid | 5.03 | | | | | |
| OPADRY | 2.16 | 24.0 | 18.9 | 15.4 | 13.9 | 18.3 |
| Clear | | | | | | |
| sodium cholate | 10.34 | 293.8 | 142.7 | 189.0 | 171.0 | 138.4 |
| sodium | 2.84 | 115.2 | 55.9 | 74.1 | 67.1 | 54.2 |
| hydroxide | | | | | | |
| sodium | 3.49 | 99.1 | 48.1 | 63.7 | 57.6 | 46.6 |
| phosphate | | | | | | |
| heptahydrate | | | | | | |
| talc | | | | 30 | 72.2 | |
| triethyl citrate | | | | 15 | 13.6 | |
| EUDRAGIT | | | | 150 | 135.7 | |
| L30D-55 | | | | | | |
| EUDRAGIT | | | | | 150 | |
| RS30D | | | | | | |
| SURELEASE | | | | | | 210.7 |
| acetyl tributyl | | | | | 30.0 | |
| citrate | | | | | | |

*Formula 1 (first sample) is in weight %, whereas each of formula 1 (second sample) through formula 3 (only sample) is in grams.

### Example D

Dosing of Dogs. For the various formulae of the EMCOMPRESS and HIM2 polydisperse from Example C, formula 1 (first sample) was not dosed in dogs. However, each of the remaining formula 1 (second sample), formula 1 (third sample), formula 2 (first sample), formula 2 (second sample), and formula 3 was manually filled into hard gelatin capsules to deliver a set amount of insulin for dosing in Beagle dogs. Each respective formula was filled into size 0 and size 00 white, opaque hard gelatin capsule shells (CAPSUGEL) at fill weights ranging from about 382 mg to about 680 mg. Use of the gelatin capsules obviated the problem of fracturing the coating(s) that may have occurred if the particles were compressed into tablets.

The study was a one-way screening study with no crossover using different respective groups of 4 dogs each that had fasted. The various capsules for each respective formula were administered orally as a single dose of 0.5 mg of insulin per kg of body weight to each of the 4 dogs in the respective group. Blood glucose was measured using the well known Eagle Glucose (Hexokinase) Procedure, which is based on a modification of the method reported by M. W. Slein in “Methods of Enzymatic Analysis, Academic Press, New York (1963).

In the Eagle Glucose (Hexokinase) Procedure, glucose is phosphorylated with ATP in a reaction catalyzed by HK. The G6P product formed is then oxidized with concomitant reduction of NAD to NADH in a reaction catalyzed by G6PDH. Formation of NADH causes an increase in absorbance at 340 nm on a spectrophotometer or colorimeter calibrated at 340 nm. For humans, the expected serum/plasma level of blood glucose ranges from 70 to 110 mg/100 ml.

More specifically for the dog dosing tests, activity was measured by a physiological response as a function of time by taking a blood sample from each dog at set time intervals in order to monitor the change up or down in the glucose level over time. Blood was drawn before dosing for all dogs. Then for some dogs, blood was drawn at 15, 30, 60, and 120 minutes post dose. For other dogs, blood was drawn every 15 minutes starting at 30 minutes post dose and through 4 hours post dose, and then drawn every 30 minutes from 4½ hours through 6 hours post dose. The plasma glucose level in the blood was measured and reported as grams of glucose per 100 ml of blood, for the initial drawing of blood at zero time, and then as a percent of the zero time value.

The results for each of formula 1 (second sample), formula 1 (third sample), and formula 2 (first sample) are summarized respectively in Table D1, Table D2, and Table D3 below. Each of these formulae that was tested in dogs performed well when the dogs were dosed, as the blood sample of the dogs showed an appropriate change in glucose level.

TABLE D1
EMCOMPRESS and HIM2 polydisperse formula 1
(second sample)

Plasma glucose level*

| Time (minutes) | dog 1 | dog 2 | dog 3 | dog 4 |
|---|---|---|---|---|
| 0 | 85 | 87 | 94 | 96 |
| 15 | 85 | 81 | 88 | 102 |
| 30 | 89 | 44 | 87 | 94 |
| 60 | 47 | 76 | 80 | 98 |
| 120 | 89 | 88 | 73 | 84 |

TABLE D2
EMCOMPRESS and HIM2 polydisperse formula 1
(third sample)

Plasma glucose level*

| Time (minutes) | dog 1 | dog 2 | dog 3 | dog 4 |
|---|---|---|---|---|
| 0 | 87 | 89 | 93 | 101 |
| 30 | 65 | 38 | 51 | 107 |
| 45 | 60 | 51 | 96 | 114 |
| 60 | 88 | 78 | 90 | 115 |
| 75 | 100 | 84 | 97 | 109 |
| 90 | 101 | 79 | 91 | 111 |
| 105 | 118 | 88 | 102 | 109 |
| 120 | 104 | 79 | 106 | 104 |
| 135 | 104 | 70 | 106 | 107 |
| 150 | 107 | 84 | 101 | 109 |
| 165 | 97 | 87 | 94 | 109 |
| 180 | 110 | 82 | 93 | 92 |
| 195 | 104 | 83 | 94 | 109 |
| 210 | 96 | 90 | 83 | 101 |
| 225 | 95 | 83 | 89 | 96 |
| 240 | 95 | 83 | 82 | 104 |
| 270 | 887 | 78 | 90 | 99 |
| 300 | 91 | 81 | 97 | 102 |
| 330 | 104 | 84 | 88 | 109 |
| 360 | 96 | 73 | 99 | 109 |

*Glucose concentration at zero time is given as mg/100 ml, and concentrations at all other times are expressed as % of the zero time value.

TABLE D3
EMCOMPRESS and HIM2 polydisperse formula 2
(first sample)

Plasma glucose level*

| Time (minutes) | dog 1 | dog 2 | dog 3 | dog 4 |
|---|---|---|---|---|
| 0 | 104 | 94 | 90 | 96 |
| 30 | 68 | 90 | 99 | 112 |
| 45 | 94 | 46 | 110 | 90 |
| 60 | 100 | 67 | 108 | 87 |
| 75 | 101 | 89 | 100 | 96 |
| 90 | 114 | 108 | 91 | 100 |
| 105 | 110 | 101 | 105 | 107 |
| 120 | 105 | 101 | 102 | 94 |
| 135 | 105 | 107 | 101 | 98 |
| 150 | 98 | 106 | 102 | 110 |
| 165 | 110 | 107 | 106 | 96 |
| 180 | 101 | 106 | 101 | 103 |
| 195 | 111 | 104 | 103 | 105 |
| 210 | 105 | 106 | 100 | 99 |
| 225 | 101 | 99 | 95 | 94 |
| 240 | 103 | 106 | 92 | 96 |
| 270 | 116 | 106 | 105 | 93 |
| 300 | 105 | 103 | 100 | 104 |
| 330 | 103 | 107 | 95 | 101 |
| 360 | 95 | 99 | 96 | 100 |

*Glucose concentration at zero time is given as mg/100 ml, and concentrations at all other times are expressed as % of the zero time value.

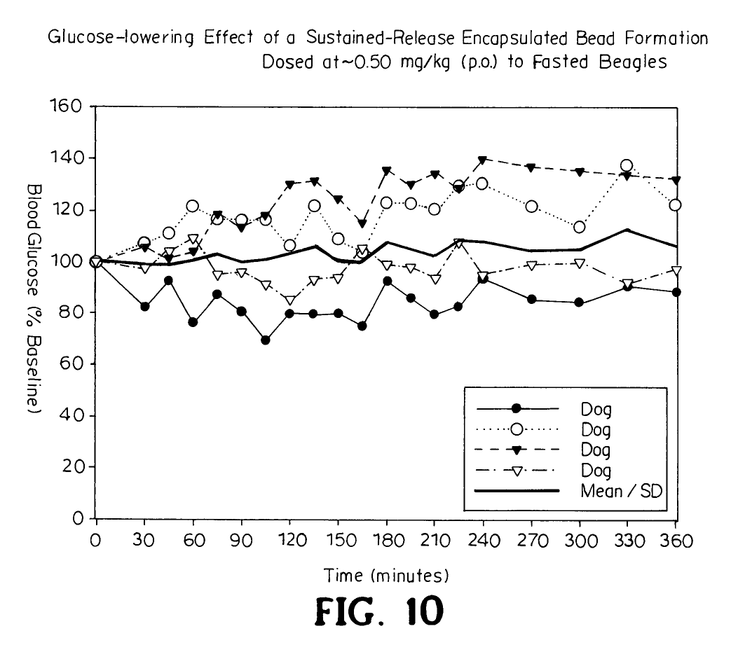

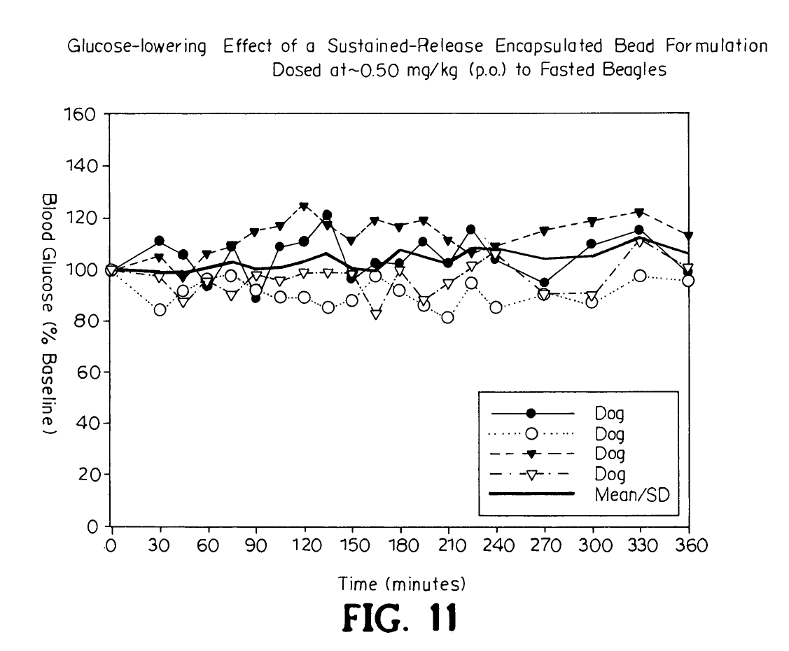

The results for each of formula 2 (second sample) and formula 3 (only sample) are summarized by the respective graphs in FIG. 10 and FIG. 11. As can be seen, the curves for each graph were rather level, which is indicative of not much of a change in blood glucose level, so that formula 2 (second sample) and formula 3 (only sample) did not perform as well in the dosed dogs as the formulae tabulated above in Table D1 which depicts formula 1 (second sample), Table D2 which depicts formula 1 (third sample), and Table D3 which depicts formula 2 (first sample).

It is believed that this performance for the graph in FIG. 10, which depicts formula 2 (second sample), was not as optimal as had been expected for the following reason. Formula 2 (second sample) had two controlled release coatings (both EUDRAGIT L30D-55 and EUDRAGIT RS30D), whereas formula 2 (first sample) of Table D3 had only one controlled release coating (EUDRAGIT L30D-55).

It is believed that this performance for the graph in FIG. 11, which depicts formula 3 (only sample), was not as optimal as had been expected for the following reason. Although formula 3 (only sample) had a lesser amount of HIM2 polydisperse insulin like formula 1 (third sample) of Table D2, formula 3 (only sample) also had a sustained release coating (SURELEASE), whereas formula 1 (third sample) of Table D2 had no such coating.

Also, it is believed that the performance was not as optimal as had been expected since dosing of a medicament in dogs, particularly for a medicament having an enteric coating, a sustained release coating, and the like, does not necessarily correlate with dosing of that same medicament in humans. The reason is that a dog's alimentary canal may simply allow the coated medicament to pass right through and out with the feces, whereas a human's alimentary canal will allow the medicament to take effect, particularly, a human's alimentary canal will attack such enteric and/or sustained release coatings, thus allowing the medicament to take effect.

### Example E

At a laboratory other than applicants' laboratory, CELLETS (trade name for microcrystalline cellulose from Glatt) was employed as the particulate substrate.

Various samples with HIM2 polydisperse insulin were prepared similar to those of Example C (i.e., in Example C, EMCOMPRESS dicalcium diphosphate dihydrate was employed as the particulate substrate). Each of the samples with CELLETS as the particulate substrate was filled into gelatin capsules and dosed in 4 dogs, followed by measuring the blood glucose levels using the Eagle Glucose (Hexokinase) Procedure similar to the measuring in Example D (where EMCOMPRESS dicalcium diphosphate dihydrate was employed as the particulate substrate).

The blood glucose results for the samples with CELLETS as the particulate substrate were substantially similar to those of Example D.

### Example F

For this example, ingredients were compressed into tablets, instead of being filled into hard gelatin capsules.

At a laboratory other than applicants' laboratory, both AVICEL PH 102 (microcrystalline cellulose) and EMCOMPRESS (dicalcium diphosphate dihydrate) together were employed as the particulate substrate.

Various samples with HIM2 polydisperse insulin (in particular, the polydisperse insulin was denoted as HIM2-PEG7, as it was uncorrected for protein and water content, i.e., expressed as anhydrous protein content) were prepared similar to those of Example C (i.e., in Example C, only EMCOMPRESS dicalcium diphosphate dihydrate was employed as the particulate substrate), but with the following changes.

A batch of AVICEL PH 102 (5.25 g), EMCOMPRESS (25.855 g), HIM2 (0.395 g), and sodium starch glycolate (0.70 g) was mixed in a blender, thus applying a coating of HIM2 to the substrate of both AVICEL PH 102 and EMCOMPRESS.

The sodium starch glycolate is a disintegrant, the purpose of which is to help the tablet disintegrate in the gastric/intestinal media, as the disintegrant has explosive expanding properties and causes the compact tablet to rupture quickly into many smaller pieces which in turn dissolve more quickly. Thus, the disintegrant aids in tablet break-up, dissolution, and ultimately, absorption of the API.

Various other ingredients, such as PEs (sodium cholate, oleic acid, capric acid, lauric acid), basic granulating solution (10 mg of NaOH per 100 ml of purified water), and/or buffering agents were applied for 8 different samples.

Each resultant blend was screened and then dried at about 50° C. Then, a small amount of magnesium stearate and CAB-O-SIL colloidal silicon dioxide were admixed into the dried blend as a lubricant system, followed by compressing into tablets.

Also, for each of the 8 different samples, a seal coating made from a batch of OPADRY II Clear (150 g) and purified water (850 g) was applied by spraying onto the tablets. Then, an enteric coating made from a batch of EUDRAGIT L30D-55 (500 g), PEG 3350 (15 g) (PEG 3350 used to be known as PEG 4000, but the manufacturers decided that PEG 3350 was a more precise designation), talc (37.5 g), and purified water (460 g) was applied by spraying.

Thus, for each of the 8 different samples, there were two sets of tablets, each set having substantially the same ingredients for that particular sample, except the first set did not have an enteric coating but the second set did have an enteric coating.

The ingredients for the various tablets are summarized below in Table F, where the amounts are in % by weight.

TABLE F

| | All samples made from of batch of AVICEL PH 102, | | | | | | | |
|---|---|---|---|---|---|---|---|---|
| | EMCOMPRESS, and HIM2 polydisperse (and also | | | | | | | |
| INGREDIENTS (first | sodium starch glycolate dispersant) | | | | | | | |
| set of tablets with no | TABLET SAMPLE NUMBER | | | | | | | |
| enteric coating) | 1 | 2 | 3 | 4 | 5 | 6 | 7 | 8 |
| color | white | blue | red | yellow | orange | green | purple | dark red |
| sodium cholate | 3 | 3 | 3 | 3 | 6 | 6 | 6 | 6 |
| oleic acid | 1 | 1 | 5 | 5 | 1 | 1 | 5 | 5 |
| capric acid | 0.5 | 0.5 | 5 | 5 | 5 | 5 | 0.5 | 0.5 |
| lauric acid | 0.5 | 0.5 | 5 | 5 | 5 | 5 | 0.5 | 0.5 |
| granulating system of | Yes | No | Yes | No | Yes | No | Yes | No |
| NaOH in water | | | | | | | | |
| buffer | Yes | No | Yes | No | Yes | No | Yes | No |
| sodium starch glycolate | 2 | 10 | 10 | 2 | 2 | 10 | 10 | 2 |
| magnesium stearate | 2 | 2 | 2 | 2 | 2 | 2 | 2 | 2 |
| CAB-O-SIL colloidal | 1 | 1 | 1 | 1 | 1 | 1 | 1 | 1 |
| silicon dioxide | | | | | | | | |
| INGREDIENTS (same | 5.5 | 7.0 | 7.0 | 5.5 | 7.0 | 5.5 | 5.5 | 7.0 |
| but second set of tablets | pH | pH | pH | pH | pH | pH | pH | pH |
| with enteric coating at | | | | | | | | |
| designated pH) | | | | | | | | |

Except for the dark red tablets of the first set of sample 8 (no enteric coating), each sample of the tablets from the first set and the second set was dosed in a separate group of 4 dogs, followed by measuring the blood glucose levels of the dogs using the Eagle Glucose (Hexokinase) Procedure similar to the measuring in Example D (where EMCOMPRESS dicalcium diphosphate dihydrate was employed as the particulate substrate).

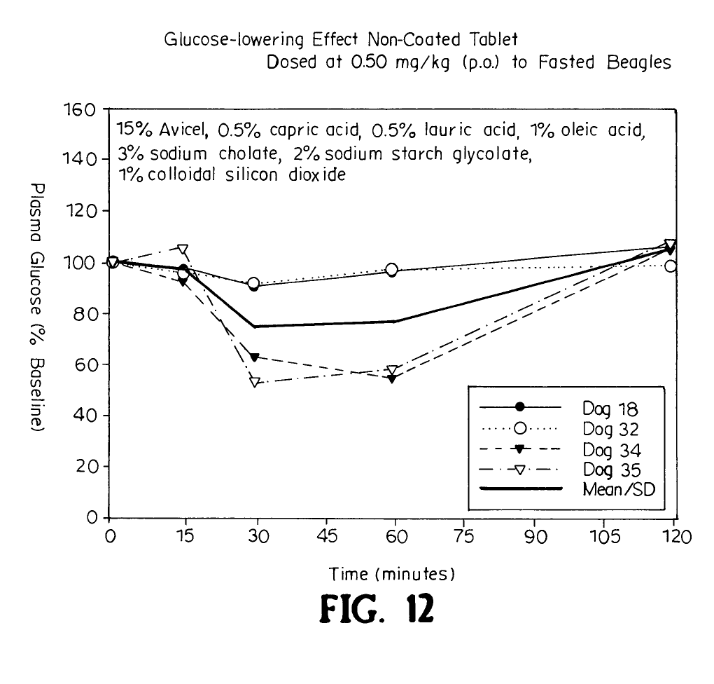

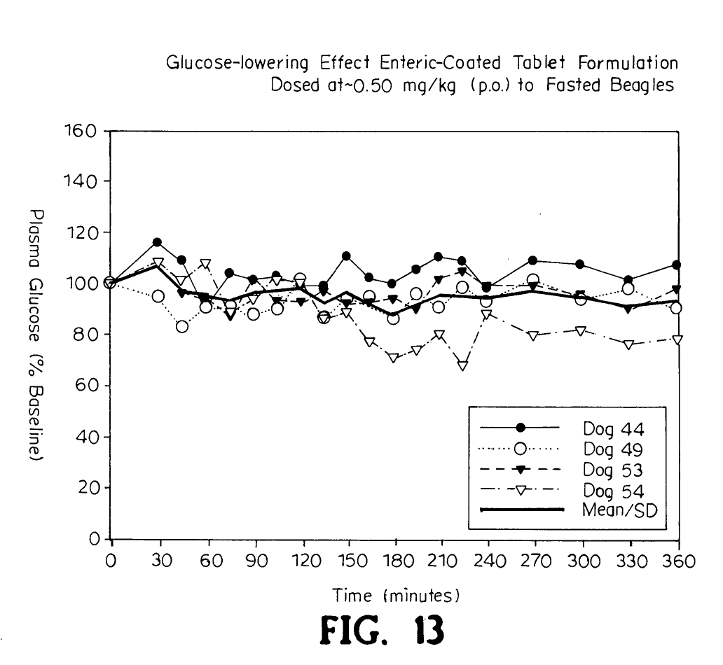

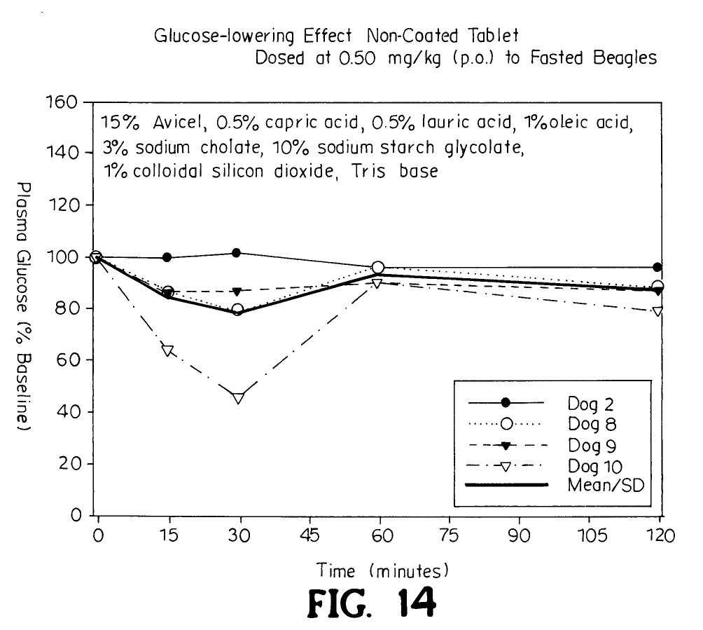

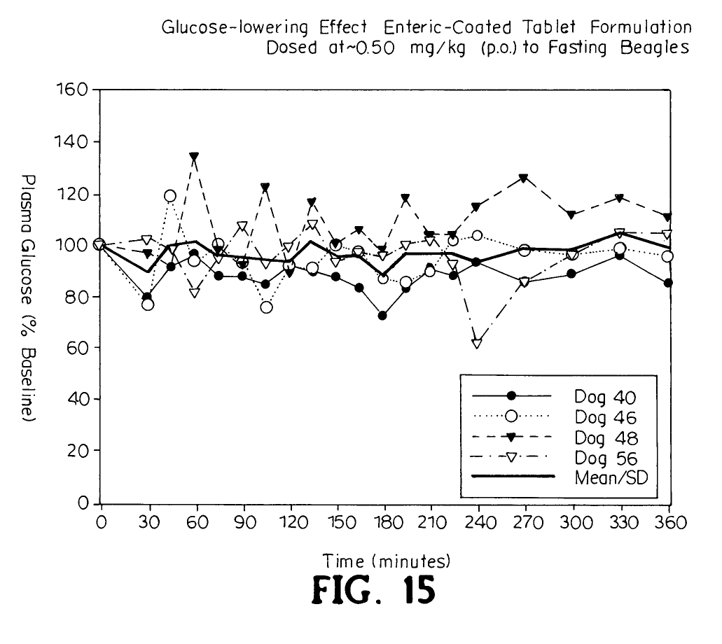

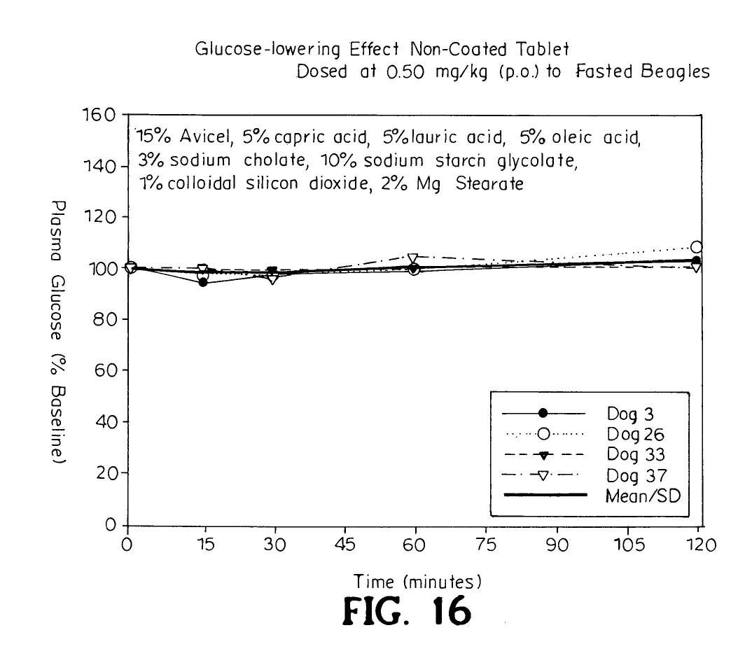

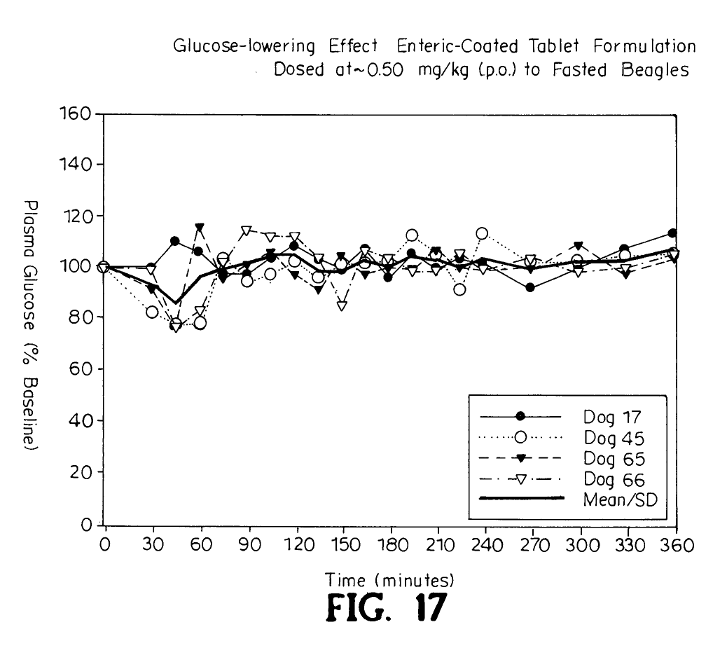

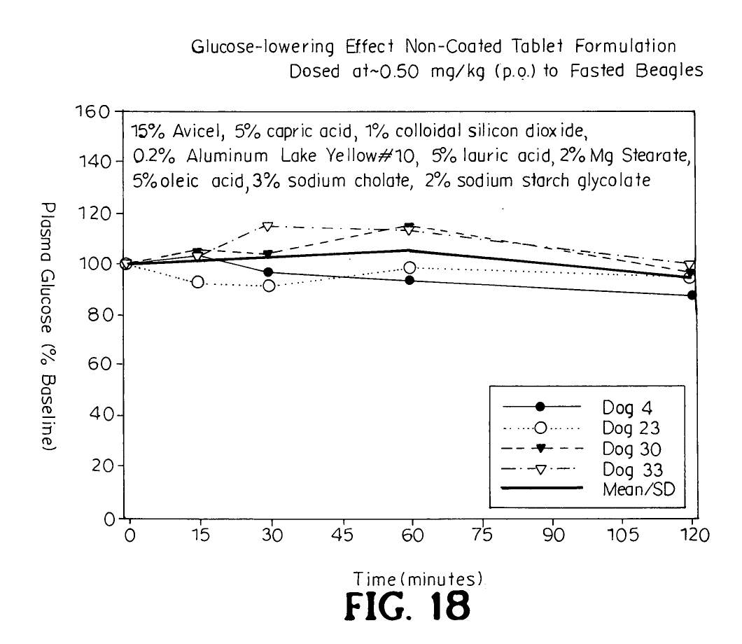

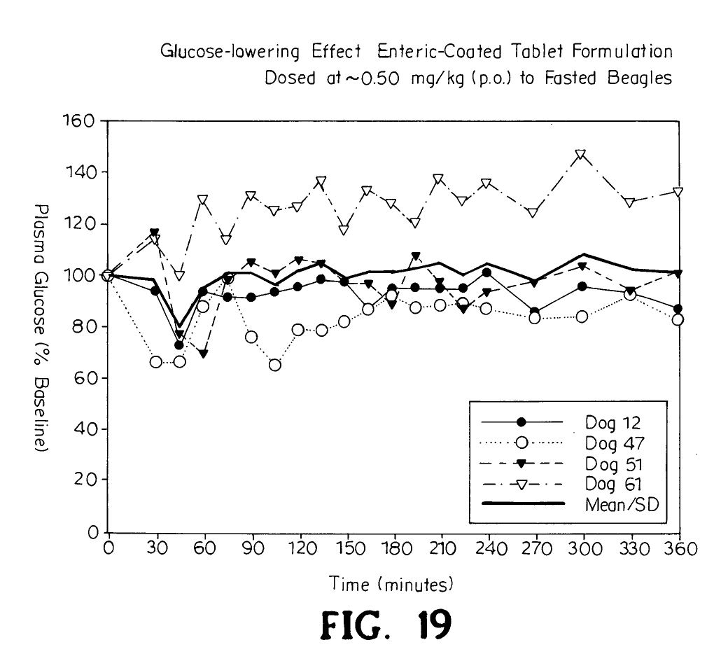

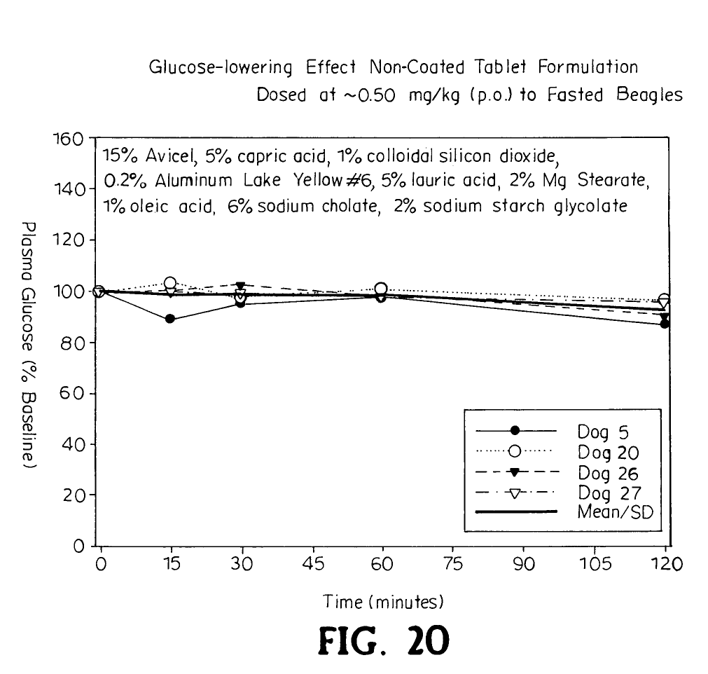

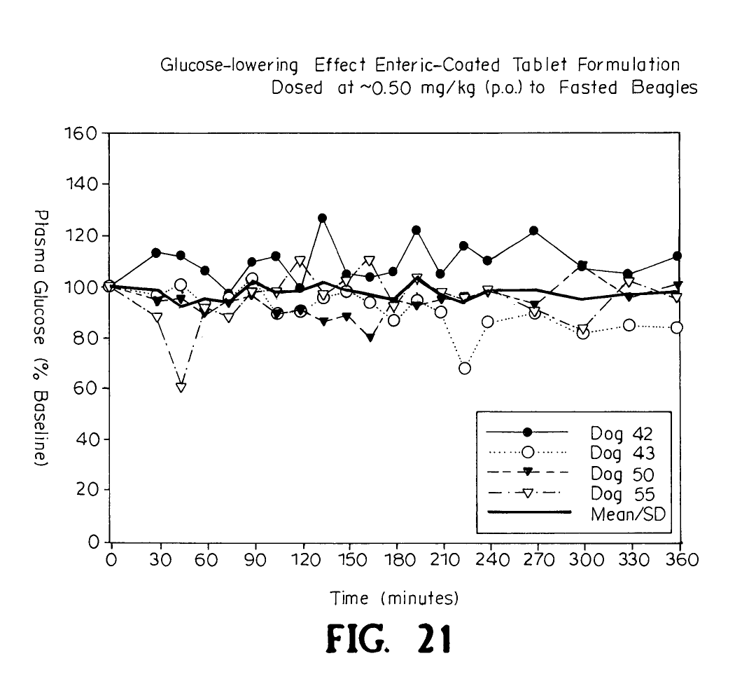

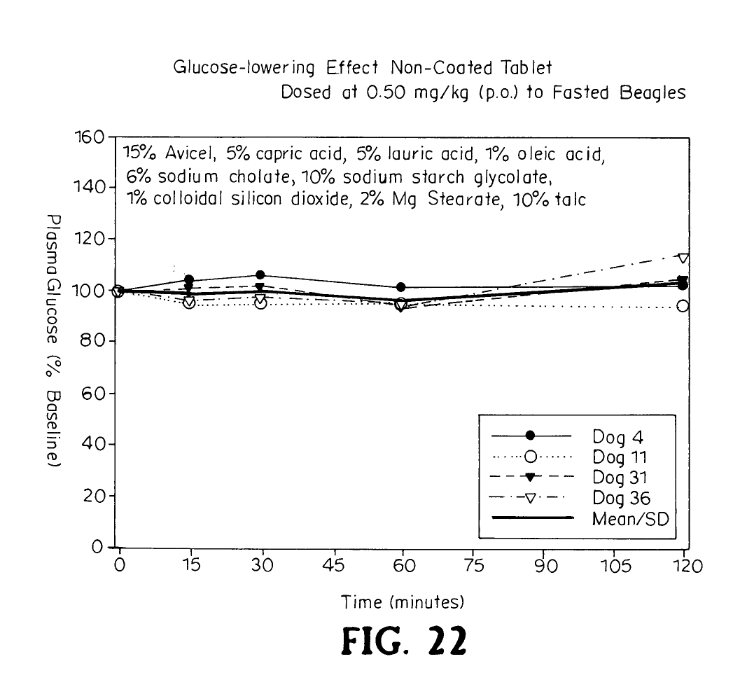

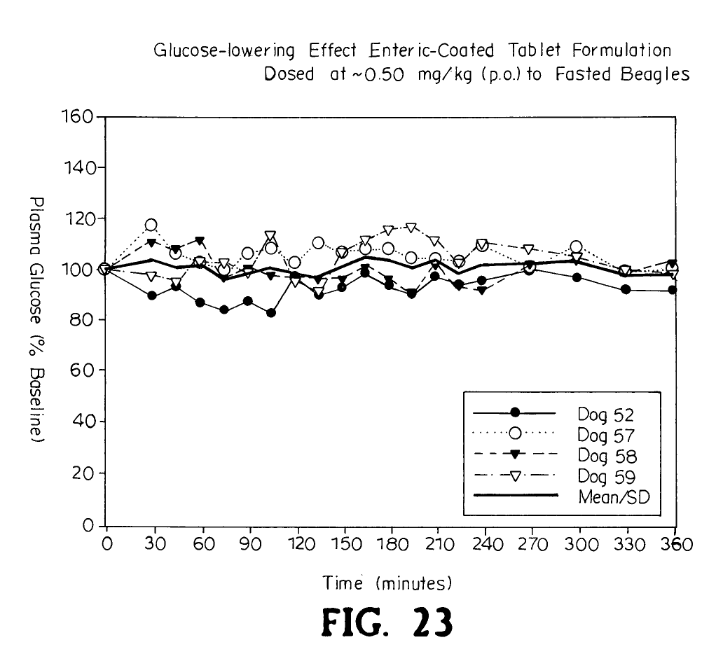

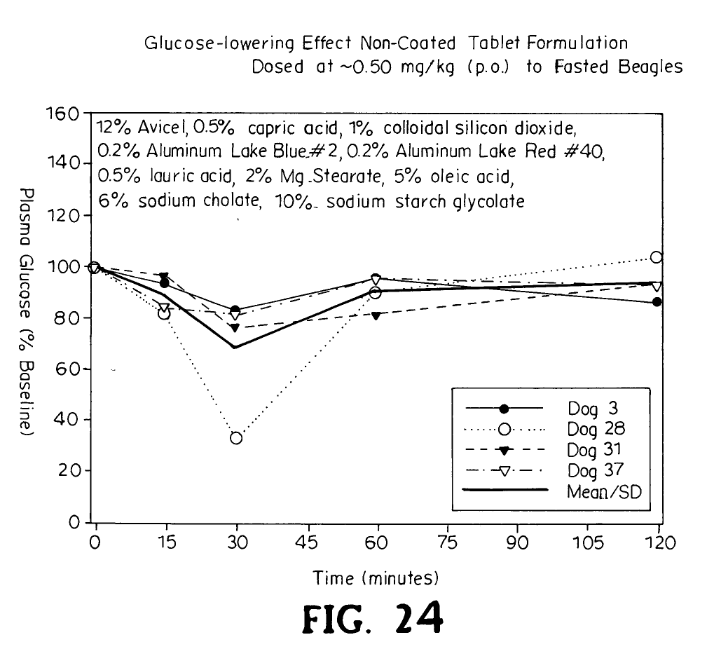

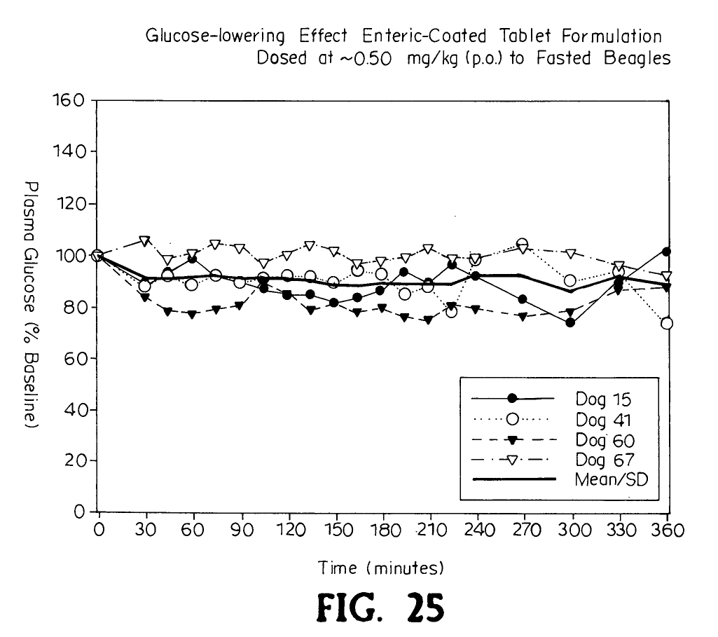

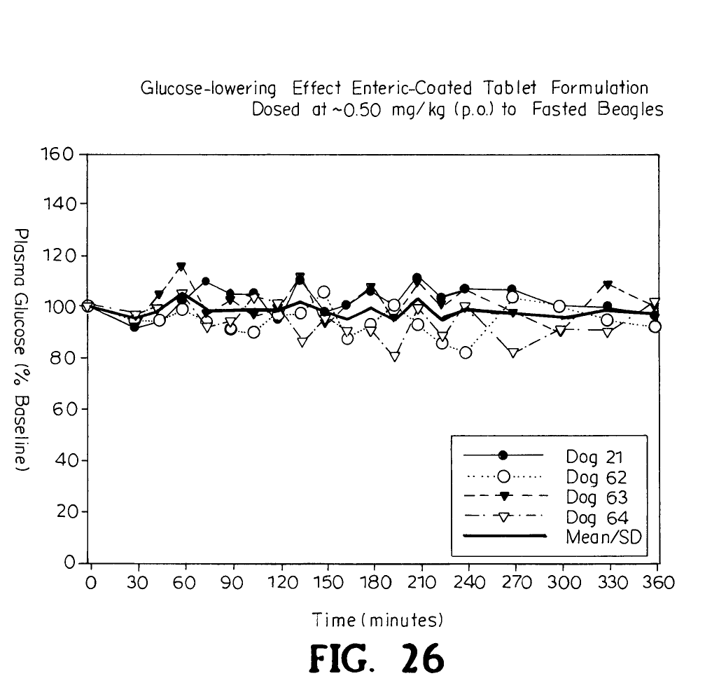

The blood glucose results for the samples were substantially similar to those of Example D. The results are summarized in FIGS. 12-26. As can be seen, some of the curves for each graph were rather level, which is indicative of not much of a change in blood glucose level. It is believed that this performance was not as optimal as had been expected for the following reason. It is noted that dosing of a medicament in dogs, particularly for a medicament having an enteric coating, a sustained release coating, and the like, does not necessarily correlate with dosing of that same medicament in humans. The reason is that a dog's alimentary canal may simply allow the medicament to pass right through and out with the feces, whereas a human's alimentary canal will allow the medicament to take effect, particularly a human's alimentary canal will attack such enteric and/or sustained release coatings, thus allowing the medicament to take effect.

Although the present invention has been shown and described in detail with regard to only a few exemplary embodiments of the invention, it should be understood by those skilled in the art that it is not intended to limit the invention to the specific embodiments disclosed. Various modifications, omissions, and additions may be made to the disclosed embodiments without materially departing from the novel teachings and advantages of the invention, particularly in light of the foregoing teachings. Accordingly, it is intended to cover all such modifications, omissions, additions, and equivalents as may be included within the spirit and scope of the invention as defined by the following claims.

What is claimed is:

1. A process for making a pharmaceutical formulation for oral administration of an active pharmaceutical ingredient comprising applying a solution of the active pharmaceutical ingredient to form a coating on a particulate pharmaceutical substrate, wherein the substrate is free of a polysaccharide.

2. The process of claim 1, wherein the active pharmaceutical ingredient comprises a peptide pharmaceutical.

3. The process of claim 1, wherein the active pharmaceutical ingredient solution includes a material selected from the group consisting of coating agents, controlled release agents, sustained release agents, pharmaceutical excipient agents, and combinations thereof.

4. The process of claim 3, wherein the agent is selected from the group consisting of colorants, film-forming polymers, plasticizers, surfactants, permeation enhancers, buffering agents, dispersions of ethyl cellulose, coating lacquers, pigments, and combinations thereof.

5. The process of claim 1, wherein applying the active pharmaceutical ingredient solution to the particulate substrate achieves an active phramaceutical ingredient load on the substrate ranging from about 0.1% to about 30% weight/weight.

6. The process of claim 1, wherein the substrate is selected from the group consisting of a calcium material, cellulose, and combinations thereof.

7. The process of claim 6, wherein the calcium material is selected from the group consisting of calcium carbonate, calcium citrate, dibasic calcium phosphate dihydrate, monobasic calcium phosphate, tribasic calcium phosphate, anhydrous dibasic calcium phosphate, calcium sulfate, calcium stearate, and combinations thereof.

8. The process of claim 1, further including applying another solution to form another coating.

9. The process of claim 8, wherein another solution is applied prior to applying the active pharmaceutical ingredient solution, after applying the active pharmaceutical ingredient solution, or a combination thereof.

10. The process of claim 8, wherein the other solution comprises a material selected from the group consisting of coating agents, controlled release agents, sustained release agents, pharmaceutical excipient agents, and combinations thereof.

11. The process of claim 10, wherein the agent is selected from the group consisting of colorants, film-forming polymers, plasticizers, surfactants, permeation enhancers, buffering agents, dispersions of ethyl cellulose, coating lacquers, pigments, and combinations thereof.

12. The process of claim 1, further including encapsulating the resultant in a gelatin capsule or compressing the resultant into a tablet.

13. A process for making a pharmaceutical formulation for oral administration of an active pharmaceutical ingredient comprising applying a solution of the active pharmaceutical ingredient to form a coating on a particulate calcium pharmaceutical substrate, wherein the substrate is free of a polysaccharide and the particulate calcium pharmaceutical substrate has been coated with a permeation enhancer.

14. The process of claim 13, further including applying another solution to form another coating, wherein the other solution comprises a material selected from the group consisting of coating agents, controlled release agents, sustained release agents, pharmaceutical excipient agents, and combinations thereof.

15. The process of claim 13, further including encapsulating the resultant in a gelatin capsule.

16. An oral pharmaceutical formulation comprising a particulate pharmaceutical substrate having an application of an active pharmaceutical ingredient coating, wherein the substrate is free of a polysaccharide.

17. The oral pharmaceutical formulation of claim 16, wherein the active pharmaceutical ingredient comprises a peptide pharmaceutical.

18. The oral pharmaceutical formulation of claim 16, wherein the active pharmaceutical ingredient coating includes a material selected from the group consisting of coating agents, controlled release agents, sustained release agents, pharmaceutical excipient agents, and combinations thereof.

19. The oral pharmaceutical formulation of claim 18, wherein the agent is selected from the group consisting of colorants, film-forming polymers, plasticizers, surfactants, permeation enhancers, buffering agents, dispersions of ethyl cellulose, coating lacquers, pigments, and combinations thereof.

20. The oral pharmaceutical formulation of claim 16, wherein the active pharmaceutical ingredient comprises an active pharmaceutical ingredient load on the substrate ranging from about 0.1% to about 30% weight/weight.

21. The oral pharmaceutical formulation of claim 16, wherein the substrate is selected from the group consisting of a calcium material, cellulose, and combinations thereof.

22. The oral pharmaceutical formulation of claim 21, wherein the calcium material is selected from the group consisting of calcium carbonate, calcium citrate, dibasic calcium phosphate dihydrate, monobasic calcium phosphate, tribasic calcium phosphate, anhydrous dibasic calcium phosphate, calcium sulfate, calcium stearate, and combinations thereof.

23. The oral pharmaceutical formulation of claim 16, further including another coating.

24. The oral pharmaceutical formulation of claim 23, wherein another coating is under the active pharmaceutical ingredient coating, over the active pharmaceutical ingredient coating, or a combination thereof.

25. The oral pharmaceutical formulation of claim 23, wherein the other coating comprises a material selected from the group consisting of coating agents, controlled release agents, sustained release agents, pharmaceutical excipient agents, and combinations thereof.

26. The oral pharmaceutical formulation of claim 25, wherein the agent is selected from the group consisting of colorants, film-forming polymers, plasticizers, surfactants, permeation enhancers, buffering agents, dispersions of ethyl cellulose, coating lacquers, pigments, and combinations thereof.

27. The oral pharmaceutical formulation of claim 16, wherein the particulate pharmaceutical substrate having an application of an active pharmaceutical ingredient coating is encapsulated in a gelatin capsule or is compressed into a tablet.

28. A process for making a pharmaceutical formulation for oral administration of an active pharmaceutical ingredient comprising applying a solution of the active pharmaceutical ingredient to form a coating on a particulate dibasic calcium phosphate dihydrate pharmaceutical substrate, wherein:
(a) the application of active pharmaceutical ingredient achieves an active pharmaceutical ingredient load on the substrate ranging from about 0.1% to 30% weight/weight, and
(b) the substrate is free of a polysaccharide and has been coated with a permeation enhancer.

29. An oral pharmaceutical formulation comprising a particulate dibasic calcium phosphate dihydrate pharmaceutical substrate having an application of an active pharmaceutical ingredient coating, wherein:
(a) the active pharmaceutical ingredient is present in a load on the substrate ranging from about 0. 1% to 30% weight/weight, and
(b) the substrate is free of a polysaccharide and has been coated with a permeation enhancer.

30. A process for making a pharmaceutical formulation for oral administration of an insulin comprising applying a solution of the insulin to form a coating on a particulate pharmaceutical substrate.

31. The process of claim 30, wherein the insulin is selected from the group consisting of human insulin, animal insulin, and combinations thereof.

32. The process of claim 31, wherein the human insulin comprises hexyl insulin monoconjugate-2-polydisperse.

33. The process of claim 30, wherein the insulin solution includes a material selected from the group consisting of coating agents, controlled release agents, sustained release agents, pharmaceutical excipient agents, and combinations thereof.

34. The process of claim 33, wherein the agent is selected from the group consisting of colorants, film-forming polymers, plasticizers, surfactants, permeation enhancers, buffering agents, dispersions of ethyl cellulose, coating lacquers, pigments, and combinations thereof.

35. The process of claim 30, wherein applying the insulin solution to the particulate substrate achieves an insulin load on the substrate ranging from about 0.1% to about 30% weight/weight.

36. The process of claim 30, wherein the substrate is selected from the group consisting of a calcium material, cellulose, and combinations thereof.

37. The process of claim 36, wherein the calcium material is selected from the group consisting of calcium carbonate, calcium citrate, dibasic calcium phosphate dihydrate, monobasic calcium phosphate, tribasic calcium phosphate, anhydrous dibasic calcium phosphate, calcium sulfate, calcium stearate, and combinations thereof.

38. The process of claim 30, further including applying another solution to form another coating.

39. The process of claim 38, wherein another solution is applied prior to applying the insulin solution, after applying the insulin solution, or a combination thereof.

40. The process of claim 38, wherein the other solution comprises a material selected from the group consisting of coating agents, controlled release agents, sustained release agents, pharmaceutical excipient agents, and combinations thereof.

41. The process of claim 40, wherein the agent is selected from the group consisting of colorants, film-forming polymers, plasticizers, surfactants, permeation enhancers, buffering agents, dispersions of ethyl cellulose, coating lacquers, pigments, and combinations thereof.

42. The process of claim 30, further including encapsulating the resultant in a gelatin capsule or compressing the resultant into a tablet.

43. A pharmaceutical formulation for oral administration of insulin comprising a particulate pharmaceutical substrate having an application of an insulin coating, wherein the particulate pharmaceutical substrate is free of a polysaccharide.

44. The oral pharmaceutical formulation of claim 43, wherein the insulin coating includes a material selected from the group consisting of coating agents, controlled release agents, sustained release agents, pharmaceutical excipient agents, and combinations thereof.

45. The oral pharmaceutical formulation of claim 44, wherein the agent is selected from the group consisting of colorants, film-forming polymers, plasticizers, surfactants, permeation enhancers, buffering agents, dispersions of ethyl cellulose, coating lacquers, pigments, and combinations thereof.

46. The oral pharmaceutical formulation of claim 43, wherein the insulin comprises an insulin load on the substrate ranging from about 0.1% to about 30% weight/weight.

47. The oral pharmaceutical formulation of claim 43, wherein the substrate is selected from the group consisting of a calcium material, cellulose, and combinations thereof.

48. The oral pharmaceutical formulation of claim 47, wherein the calcium material is selected from the group consisting of calcium carbonate, calcium citrate, dibasic calcium phosphate dihydrate, monobasic calcium phosphate, tribasic calcium phosphate, anhydrous dibasic calcium phosphate, and combinations thereof.

49. The oral pharmaceutical formulation of claim 43, further including another coating.

50. The oral pharmaceutical formulation of claim 49, wherein another coating is under the insulin coating, over the insulin coating, or a combination thereof.

51. The oral pharmaceutical formulation of claim 49, wherein the other coating comprises a material selected from the group consisting of coating agents, controlled release agents, sustained release agents, pharmaceutical excipient agents, and combinations thereof.

52. The oral pharmaceutical formulation of claim 51, wherein the agent is selected from the group consisting of colorants, film-forming polymers, plasticizers, surfactants, permeation enhancers, buffering agents, dispersions of ethyl cellulose, coating lacquers, pigments, and combinations thereof.

53. The oral pharmaceutical formulation of claim 43, wherein the particulate pharmaceutical substrate having an application of an insulin coating is encapsulated in a gelatin capsule or is compressed into a tablet.

54. A process for making a pharmaceutical formulation for oral administration of an insulin comprising applying a solution of an insulin to form a coating on a particulate dibasic calcium phosphate dihydrate pharmaceutical substrate, wherein:
(a) the application of the insulin achieves an insulin load on the substrate ranging from about 0.1% to 30% weight/weight, and
(b) the substrate is free of a polysaccharide and has been coated with a permeation enhancer.

55. An oral pharmaceutical formulation of insulin comprising a particulate dibasic calcium phosphate dihydrate pharmaceutical substrate having an application of an insulin coating, wherein:
(a) the insulin is present in a load on the substrate ranging from about 0.1% to 30% weight/weight, and
(b) the substrate is free of a polysaccharide and has been coated with a permeation enhancer.
